(19) United States
(12) Patent Application Publication
(10) Pub. No.: US 2022/0249140 A1
(43) Pub. Date: Aug. 11, 2022

(54) TRANSITION REGION IN INTRAMEDULLARY PORTION OF PROSTHESIS

(71) Applicant: Biomet Manufacturing, LLC, Warsaw, IN (US)

(72) Inventors: Philippe Favre, Zurich (CH); Stephen Kays, Warsaw, IN (US)

(21) Appl. No.: 17/669,151

(22) Filed: Feb. 10, 2022

Related U.S. Application Data
(60) Provisional application No. 63/148,349, filed on Feb. 11, 2021.

(51) Int. Cl. A61B 17/72; A61B 17/74

(52) U.S. Cl. CPC A61B 17/7266; A61B 17/746

(57) ABSTRACT

The present disclosure provides an orthopedic implant that can include an intramedullary stem transition region that fits in the bony intramedullary canal. The transition region can run as a monolithic piece between the implant body and the intramedullary stem, with a squared cross-section away from the implant body and a circular cross-section close to the stem body. The transition region can allow for higher implant strength with reduced rotation for the implant.

### CLAIM OF PRIORITY

This application claims the benefit of U.S. Provisional Patent Application Ser. No. 63/148,349, filed on Feb. 11, 2021, the benefit of priority of which is claimed hereby, and which is incorporated by reference herein in its entirety.

### BACKGROUND

The present invention relates generally to devices for arthroplasty and bone segment replacement and specifically to intramedullary implants and stems.

Orthopedic implants can include medical devices manufactured to replace a missing joint or bone or to support a damaged bone. Orthopedic implants can include hip, knee, elbow, shoulder, and other types of implants. Orthopedic implants can include nails, plates, or other types of fixations or replacements as needed.

Artificial joints can include ball and socket or hinge joints designed to match as closely as possible the function of the natural joint. To duplicate a joint's natural motion, a total joint replacement implant can include a bearing surface component, such as a spherical ball to replace the head of the femur, and a component which fits into the intramedullary canal of the femur, tibia, or humerus to provide stability for the bearing surface. The stem component may also be used in the replacement of segments of the long bones of the upper or lower extremity.

Intramedullary stems, rods, or nails, used in the intramedullary canal, can be used to provide stability to a long bone in the body. Intramedullary stems can extend from implant bodies to better secure the implant, such as a hip or knee replacement, into the body by securing the intramedullary stem into the intramedullary canal of a proximal bone.

### SUMMARY OF THE DISCLOSURE

The present disclosure provides an orthopedic implant that can include an intramedullary stem transition region that fits in the bony intramedullary canal. The transition region can run as a monolithic piece between the implant body and the intramedullary stem, with a squared cross-section away from the implant body and a circular cross-section close to the stem body. The transition region can allow for a higher implant strength with reduced axial rotation for the implant.

Orthopedic implants can include both a body segment, that replaces a joint, and an intramedullary stem, that helps to fix the implant to the bone canal of the targeted bone. The intramedullary stem can have a small cross-section so that the intramedullary stem fits into the target bone canal. When a load is applied to the implant, bending in the intramedullary stem can cause peak stress near the intersection of the implant body and the intramedullary stem.

Conventionally, intramedullary stems have square cross sections. A square-shaped cross-section can help provide rotational stability when inserted into the intramedullary canal of the targeted bone. For example, a square cross-section can reduce undesired axial rotation of the implant once inserted. The intramedullary stem should be sized, shaped, or arranged for insertion into the intramedullary canal, and thus should fit within the bony intramedullary canal.

However, for a given intramedullary canal size, it is more advantageous for the strength of the implant to fill the intramedullary canal with the intramedullary stem. For example, a circular cross-section intramedullary stem would better fill the intramedullary canal and create a stronger, more robust implant. In this case, more material is present in the intramedullary stem to allow for a higher strength and more dissipation of stress along the intramedullary stem compared to a square-shaped cross-section.

Disclosed herein, an intramedullary stem can have both a square-shaped cross section and a circular-shaped cross section, joined by a transition region. The squared cross-section can be further away from the implant body, and the circular cross-section can be closer to the implant body. The squared cross-section can allow for rotational stability, while the circular cross-section can allow for increased strength.

The proposed intramedullary stem with a transition segment can have a lesser chance of fracture compared to designs that only includes a squared cross-section. The transition segment allows for lower stress in the implant between the implant body and the intramedullary stem.

Conversely, a design with only a cylindrical cross-section can risk device rotation within the bone canal, which may lead to prosthesis loosening or migration. Having an intramedullary stem with a transition segment including both squared and circular cross-sections can prevent this potential rotation.

In an example, an orthopedic implant can include a body segment for replacing a joint and a stem segment for insertion into a bone canal and fixation of the implant therein, the stem segment comprising a first portion having a circular cross section, and a second portion having a squared cross section.

In an example, an orthopedic implant can include a body segment for replacing a joint; and a stem segment for insertion into a bone canal and fixation of the implant therein, the stem segment comprising a first portion having a circular cross section, and a second portion having a rectangular cross section, wherein the stem segment is a monolithic piece, wherein the first portion is distal of the second portion in the stem segment, the first portion has a strength of about 1.5 to 2.5 times compared to the second portion, and wherein the stem segment comprises a ratio of the first portion to the second portion of about 1:1 to about 1:5.

In an example, a method of implanting an intramedullary stem into a patient's bone can include: preparing the patient's intramedullary canal to receive a stem; providing a stem comprising a first portion having a circular cross section, and a second portion having a squared cross section, wherein the stem segment is a monolithic piece; and inserting the stem into the patient's intramedullary canal.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIGS. 1A-1B illustrate views of a prior art orthopedic implant with a stem having a circular cross-section.

FIG. 2 illustrates a view of an orthopedic implant with a transition region.

FIG. 3 illustrates a schematic drawing of an orthopedic implant with a transition region.

FIG. 4 illustrates stress plots for various cross-sections used for an intramedullary stem.

### DETAILED DESCRIPTION

The present disclosure describes, among other things, an orthopedic implant having a body portion and an intramedullary stem portion. The intramedullary stem portion can include a distal section having a squared cross-section and a proximal section having a circular cross-section, connected by a transition region.

The intramedullary implant stem component of the present invention can allow for intramedullary fixation when assembled with appropriate joint replacement implants such as knee, hip, and shoulder prosthesis. In some cases, the intramedullary implant stem component may be used in the replacement of segments of the long bones of the upper or lower extremity.

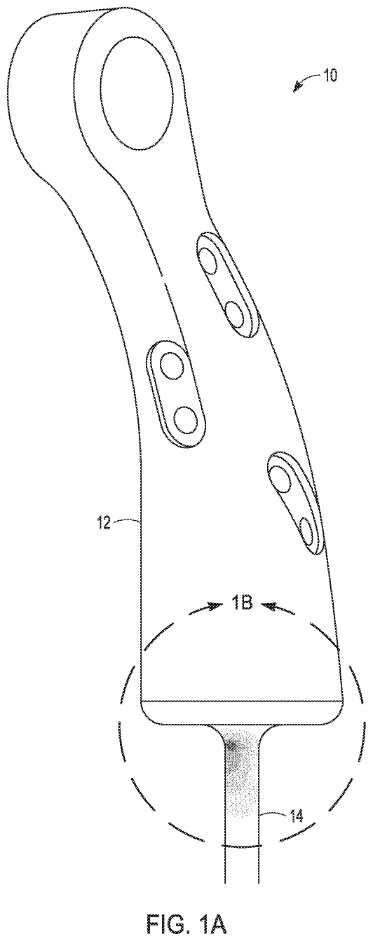

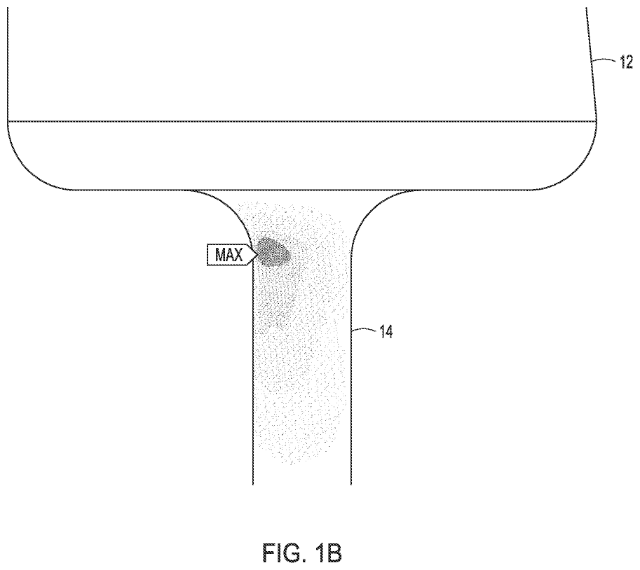

FIGS. 1A-1B illustrate views of a prior art orthopedic implant 10 with a stem having a squared cross-section. The orthopedic implant 10 can have a body portion 12 and an intramedullary stem 14. FIG. 1B shows zoomed in portion C of the implant 10, with peak stress location (“MAX”).

Orthopedic implant 10 features the body portion 12 that replaces the joint in a patient, and the intramedullary stem 14, that provides fixation into a bone canal of the patient. The intramedullary stem 14 can have a small cross-section to fit into the host bone. Under load, bending induces peak stress near the intersection of the body and intramedullary stem at the peak stress location. This can occur with conventionally shaped intramedullary stems, such as those with squared cross-sections. The peak stress location can cause tension in the implant 10 and potential fragility or issues after implementation.

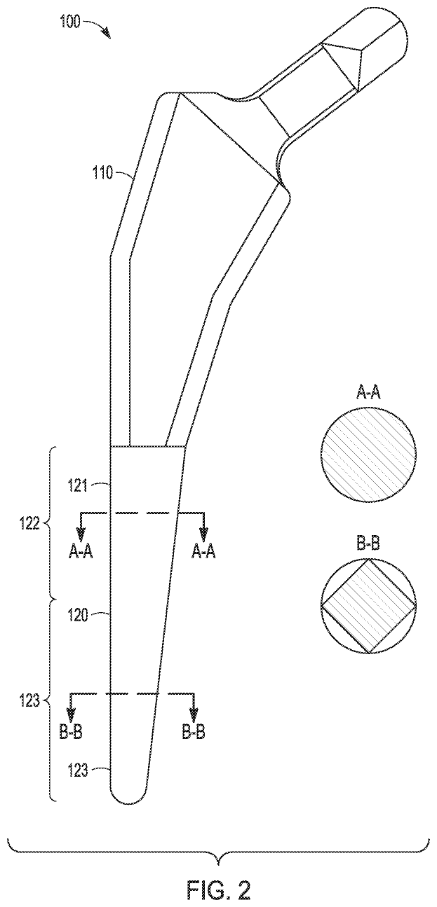

In some cases, modular designs have been used with both squared and circular portions, but in modular designs, the circular region is motivated by the need for a circular taper near the joint, to allow for modularity. These modular designs do not include a transition from squared to circular in monobloc designs in order to reduce risk of device fracture and loosening is new, such as discussed below in FIGS. 2-4.

FIG. 2 illustrates a schematic drawing of an orthopedic implant 100 with a transition region. The orthopedic implant 100 can include a body portion 110 and an intramedullary stem 120. The intramedullary stem 120 can have a first portion 122 and a second portion 124. The orthopedic implant 100 can be a medical device manufactured to replace, support, or enhance a biological structure, such as a joint. For example, the implant 100 can be a hip, knee, elbow, or other implant. Shown in FIG. 2 is an example of a hip implant, such as for a total or hemi hip replacement. For a hip replacement, the implant 100 can be used to replace the femoral head. In some cases, an implant such as the implant 100 can be used to replace an arthritic or fractured hip joint.

Body portion 110 can support the intramedullary stem 120, such that the intramedullary stem 120 extends outwards from the body portion 110. Both the body portion 110 and the intramedullary stem 120 can extend into the bone when applied. In some cases, a head can extend from the body portion 110 opposite the intramedullary stem 120. The head can be a variety of shapes, such as a rounded or plate portion shaped, sized, or arranged to replace, reinforce, or supplement the femoral head. The body portion 110 can be used in a system including an acetabular cup, liner, or other implant component, that is implantable within a patient's acetabulum. In an example, the body portion 110 can include a prosthetic femoral head configured for articulation with a prosthetic acetabular cup. The body portion 110 can be a standard size or tailored to the specific patient based on measurements derived from medical images of the patient's joint.

The body portion 110 can include a bone on-growth or in-growth material, or have a bone on-growth or in-growth feature, such as a raised splined surface, a roughened surface, metallic beads, a grit blasted surface, a porous surface, or a hydroxyapatite coating, or combinations thereof. For example, Trabecular Metal® a highly porous biomaterial made from elemental tantalum with structural, functional, and physiological properties similar to that of bone, can be used. All or a portion of the body portion 110 can be formed from a bone on-growth or in-growth material, or the material can be applied as an external coating.

The intramedullary stem 120 can extend from the body portion 110 and be used to secure the implant 100 in the body. The intramedullary stem 120 can extend from a proximal end 121, near the body portion 110, to a distal end 123, longitudinally spaced from the body portion 110. The intramedullary stem 120 can be formed integrally or monolithically with the body portion 110. Alternatively, the intramedullary stem 120 can be formed as a separate element that is attachable to the body portion. The distal end 123 can be adapted to be inserted into a patient's intramedullary canal, and can help to guide insertion of all or a portion of the intramedullary stem 120 into the intramedullary canal. The distal end 123 can be fiat rounded, bullet-nosed, or other shapes as desired for insertion into the intramedullary canal. These shapes can allow for the distal end 123 to be easily inserted into the canal, and minimize interference with and trauma to the surrounding hone. The intramedullary stem can optionally include one or more connection portions extending from the stem, such as a tapered or screw connection. In some cases, the intramedullary stem can include one or more tapered regions, such as to allow for a closer fit to the intramedullary canal during insertion. In this case, the first portion 122, the second portion 124, or both, can be tapered according to the specific application of the implant.

The intramedullary stem 120, extending from the proximal end 121 to the distal end 123, can be straight or curved, such that it substantially matches the curvature of the anatomy of the patient to which it is being applied.

The intramedullary stem 120 can include the first portion 122, having a circular cross-section and the second portion 124 having a non-circular cross-section, such as a rectangular, squared, polygonal, or diamond-shaped cross-section. The transition in the intramedullary stem from the circular cross section to the non-circular cross-section allows both for rotational stability and improved strength of the implant. The intramedullary stem 120 can he a monolithic or monobloc piece, such that the two types of cross-sections are made of a single piece or material without additional joints or connections.

The intramedullary stem 120 can have a ratio of the first portion to the second portion of about 1:1 to about 1:5, or about 1:1 to about 1:3, depending on the length of the patient's bone, the joint being replaced, and other measurement for the procedure. The first portion can have a strength of about 1.5 to 2.5 times compared to the second portion, or a strength of about 1.6 to about 2.2 times compared to the second portion.

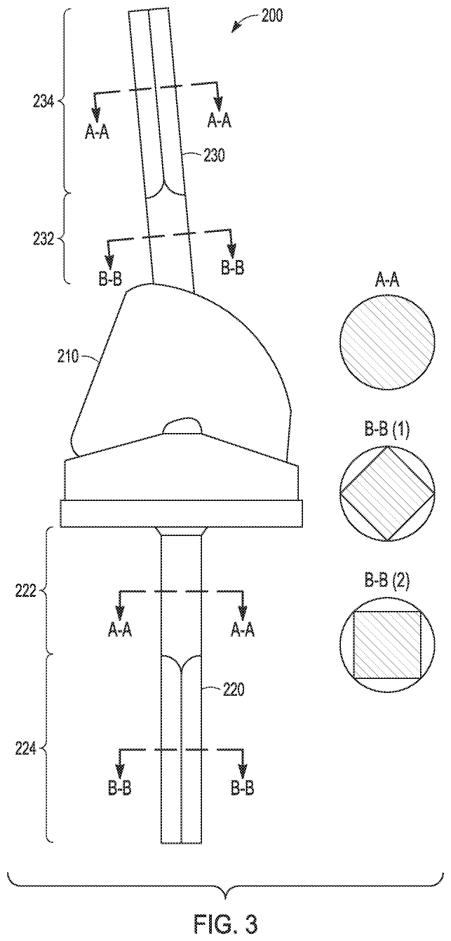

FIG. 3 illustrates a schematic drawing of an orthopedic implant 200 with intramedullary stems with cross-section transitions. The orthopedic implant 200 can include a body portion 210 and two intramedullary stems 220, 230. The first intramedullary stem can have portions 222 and 224. The second intramedullary stem 230 can have portions 232 and 234. Body portion 210 and intramedullary stems 220, 230 are similar to those discussed with reference to FIG. 2 above.

Implant 200 includes two intramedullary stems 220, 230, such as for a knee replacement, for an application requiring two intramedullary stems. In implant 200, the intramedullary stems 220, 230, have similar cross-sections to the implant 100. Each of the intramedullary stems 220, 230, can include both a squared-type crossed section closer to the body portion 210 and a circular cross-section distal of the body portion 210. In this way, the implant 200 can be both rotationally stable and strong.

The implant 200 or implant 100 can be made by a variety of machining methods. For example, the implant intramedullary stems can be made as a monobloc or monolithic piece, such as by additive manufacturing, or other techniques that do not create additional joints or bending the intramedullary stem. This can allow for a smooth transition from the rectangular cross-section to the circular cross-section of the intramedullary stem.

The implants 200 or 100 can be applied to a human joint using conventional surgical techniques. For example, the method of implanting the intramedullary stem into a patient's bone can include preparing the patients intramedullary canal to receive a stem, providing a stem comprising a first portion having a circular cross section, and a second portion having a squared cross section, wherein the stem segment is a monolithic piece, and inserting the stem into the patient's intramedullary canal. In some cases, and adhesive, such as a cement, can be used.

The bending stress on a circular cross-section compared to a squared cross-section can be theoretically modeled for an intramedullary stem to show the strength of each type of cross section. For a theoretical analysis (not accounting for radii in the corners of the squared cross-section, or flared regions in the transition between the body and the intramedullary stem), maximal bending stress (σ) is given by:

$$σ = \frac{Moment}{S} \qquad Equation 1$$

where S is the section modulus that combines the centroidal moment of inertia (Ic) and the centroidal distance (c), the section modulus is given as

$$S = \frac{Ic}{c} \qquad Equation 2$$

For a circular cross-section, the section modulus is given as:

$$S = \frac{π {d}^{3}}{32} ≅ \frac{{d}^{3}}{10} \qquad Equation 3$$

For a rectangular cross-section, the section modulus is given as:

$$S = \frac{width * {height}^{2}}{6} \qquad Equation 4$$

For a squared cross-section inscribed within an intramedullary canal of diameter d,

$$width = height = \frac{d}{\sqrt{2}} .$$

### Therefore,

$$S = \frac{{( \frac{d}{\sqrt{2}} )}^{3}}{6} ≅ \frac{{d}^{3}}{17} \qquad Equation 5$$

Comparing Equation 3 and 5 demonstrates that the section modulus in a squared cross-section is 1.7 times smaller than in a circular cross-section. From equation 1, the stress can be reduced by a factor of roughly 1.7 with a circular cross-section.

For a diamond cross-section,

$$S = \frac{width * {height}^{2}}{24} \qquad Equation 6$$

For a diamond cross-section inscribed within an intramedullary canal of diameter d, width=height=d. Therefore,

$$S = \frac{{d}^{3}}{24} \qquad Equation 7$$

that the section modulus in a squared cross-section is 2.4 times smaller than in a circular cross-section. From Equation 1, the stress can be reduced by a factor of roughly 2.4 with a circular cross-section.

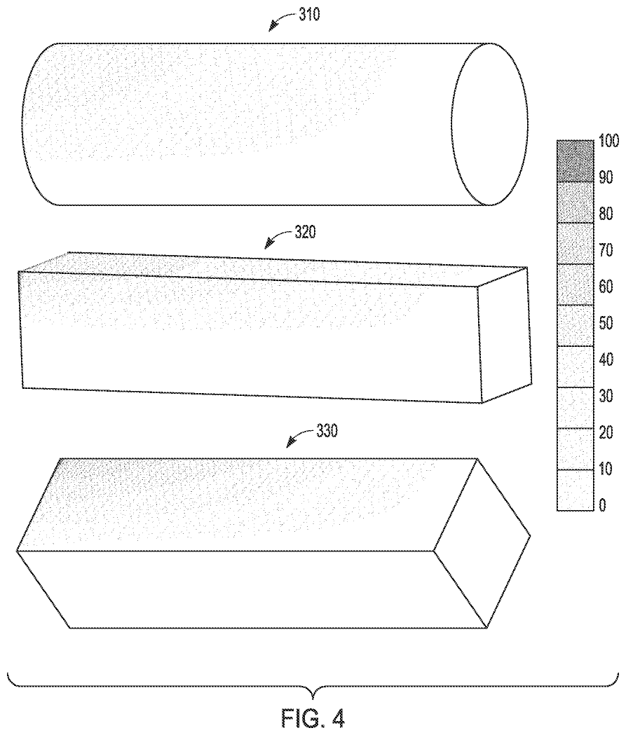

For this reason, including a circular cross section portion in the peak stress region of an intramedullary stem can reduce overall bending stress on the intramedullary stem and the implant as a whole. This mathematically modeling is exhibited in the Example, and shown and discussed with reference to FIG. 4, below.

### EXAMPLE

A series of intramedullary stem pieces of various cross-sections were tested for bending stress. FIG. 4 illustrates stress plots for various cross-sections used for an intramedullary stem, including a circular cross section 310, a rectangular cross-section 320, and a diamond (squared) cross-section 330.

In order to analyze bending stress, beams of circular, squared and diamond cross-sections were analyzed using the finite element method. The squared and diamond cross-sections were inscribed in the circular cross-section. All cross-sections were extruded to the same length. During testing, one end of the beam was held fixed in space, and the other end was loaded with a force perpendicular to the beam axis.

Bending stress was measured at a 1 mm distance away from the fixed face to avoid artifacts from this boundary condition. Stress values were 1.6 and 2.2 times higher in the squared and in the diamond cross-section respectively, when compared to the circular cross-section. Stress plots are shown in FIG. 4. These results confirm the theoretical analysis discussed with reference to FIG. 2 above.

### Various Notes & Examples

Each of these non-limiting examples can stand on its own, or can be combined in various permutations or combinations with one or more of the other examples.

Example 1 can include an orthopedic implant comprising a body segment for replacing a joint and a stem segment for insertion into a bone canal and fixation of the implant therein, the stem segment comprising a first portion having a circular cross section, and a second portion having a squared cross section.

Example 2 can include Example 1, wherein the stem segment is a monolithic piece.

Example 3 can include any of Examples 1-2, wherein the implant comprises a bone on-growth or bone in-growth feature comprising a raised splined surface, a roughened surface, metallic beads, a grit blasted surface, a porous surface, or a hydroxyapatite coating, or combinations thereof.

Example 4 can include any of Examples 1-3, wherein the first portion is distal of the second portion in the stem segment.

Example 5 can include any of Examples 1-4, wherein the stem segment is curved.

Example 6 can include any of Examples 1-5, wherein the stem segment comprises a ratio of the first portion to the second portion of about 1:1 to about 1:5.

Example 7 can include any of Examples 1-6, wherein the stem segment comprises a ratio of the first portion to the second portion of about 1:1 to about 1:3.

Example 8 can include any of Examples 1-7, wherein the first portion has a strength of about 1.5 to 2.5 times compared to the second portion.

Example 9 can include any of Examples 1-8, wherein the first portion has a strength of about 1.6 to 2.2 times compared to the second portion.

Example 10 can include any of Examples 1-9, further comprising a second stem segment opposite the first stem segment, wherein the second stem segment comprises a monobloc piece.

Example 11 can include any of Examples 1-10, wherein the second stem segment comprises a first portion having a rectangular cross-section and a second portion having a circular cross-section.

Example 12 can include any of Examples 1-11, wherein the stem segment further comprises one or more flared regions.

Example 13 can include any of Examples 1-12, further comprising a connection portion extending from the stem segment.

Example 14 can include any of Examples 1-13, wherein the connection portion comprises a taper or screw connection.

Example 15 can include any of Examples 1-14, wherein the implant is a knee, hip, shoulder, elbow, or ankle replacement.

Example 16 can include any of Examples 1-15, wherein the body segment comprises a head and a cup fitted into the head.

Example 17 can include an orthopedic implant comprising: a body segment for replacing a joint; and a stem segment for insertion into a bone canal and fixation of the implant therein, the stem segment comprising a first portion having a circular cross section, and a second portion having a rectangular cross section, wherein the stem segment is a monolithic piece, wherein the first portion is distal of the second portion in the stem segment, the first portion has a strength of about 1.5 to 2.5 times compared to the second portion, and wherein the stem segment comprises a ratio of the first portion to the second portion of about 1:11. to about 1:5.

Example 18 can include Example 17, wherein the second portion has a diamond cross-section.

Example 19 can include any of Examples 17-18, wherein the first portion has a strength of about 1.6 to 2.2 times compared to the second portion.

Example 20 can include a method of implanting an intramedullary stem into a patient's bone, comprising: preparing the patient's intramedullary canal to receive a stem; providing a stem comprising a first portion having a circular cross section, and a second portion having a squared cross section, wherein the stem segment is a monolithic piece; and inserting the stem into the patient's intramedullary canal.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” in this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The terms “distal” and “proximal” by definition refer to a location further from or nearer to, respectively, a reference point. The reference point may vary in different fields, e.g. medical and mechanical. For example, in the medical field, the reference point may be the body midline, or mesial plane. The reference point could also refer to a point of attachment whether the attachment is mechanical or non-mechanical. Herein, to avoid a change of meaning of these terms based on the location or orientation of the implant in the body, proximal shall refer to being next to or nearest the point of attachment, or the point at which the shaft integrally meets another element of the implant device. The reference point can be the taper or threaded connection in a modular assembly-type implant, or in a one-piece implant, the integral meeting point of the shaft with the remainder of the implant. Similarly, distal shall refer to being situated away from or furthest from the reference point.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. An orthopedic implant comprising:
a body segment; and
a stem segment for insertion into a bone canal and fixation of the implant therein, the stem segment comprising a first portion having a circular cross section, and a second portion having a non-circular cross section.

2. The implant of claim 1, wherein the stem segment is a monolithic piece.

3. The implant of claim 1, wherein the implant comprises a bone on-growth or bone in-growth feature comprising a raised splined surface, a roughened surface, metallic beads, a grit blasted surface, a porous surface, or a hydroxyapatite coating, or combinations thereof.

4. The implant of claim 1, wherein the first portion is distal of the second portion in the stem segment.

5. The implant of claim 1, wherein the non-circular cross section comprises a rectangular, square, or polygonal cross-section.

6. The implant of claim 1, wherein the stem segment comprises a ratio of the first portion to the second portion of about 1:1 to about 1:5.

7. The implant of claim 6, wherein the stem segment comprises a ratio of the first portion to the second portion of about 1:1 to about 1:3.

8. The implant of claim 1, wherein the first portion has a strength of about 1.5 to 2.5 times compared to the second portion.

9. The implant of claim 8, wherein the first portion has a strength of about 1.6 to 2.2 times compared to the second portion.

10. The implant of claim 1, further comprising a second stem segment opposite the first stem segment, wherein the second stem segment comprises a monobloc piece.

11. The implant of claim 10, wherein the second stem segment comprises a first portion having a circular cross-section and a second portion having a non-circular cross-section.

12. The implant of claim 1, wherein the stem segment further comprises one or more flared regions.

13. The implant of claim 1, further comprising a connection portion extending from the stem segment.

14. The implant of claim 13, wherein the connection portion comprises a taper or screw connection.

15. The implant of claim 1, wherein the implant is a prosthetic knee, hip, shoulder, elbow, or ankle component.

16. The implant of claim 1, wherein the body segment comprises a prosthetic femoral head.

17. An orthopedic implant comprising:
a body segment for replacing a joint;
a stem segment for insertion into a bone canal and fixation of the implant therein, the stem segment comprising a first portion having a circular cross section, and a second portion having a rectangular cross section, wherein the stem segment is a monolithic piece,
wherein the first portion is distal of the second portion in the stem segment, the first portion has a strength of about 1.5 to 2.5 times compared to the second portion, and wherein the stem segment comprises a ratio of the first portion to the second portion of about 1:1 to about 1:5.

18. The implant of claim 17, wherein the second portion has a polygonal or diamond cross-section.

19. The implant of claim 17, wherein the first portion has a strength of about 1.6 to 2.2 times compared to the second portion.

20. A method of implanting an intramedullary stem into a patient's bone, comprising:
preparing the patient's intramedullary canal to receive a stem;
providing a stem comprising a first portion having a circular cross section, and a second portion having a squared cross section, wherein the stem is a monolithic piece; and
inserting the stem into the patient's intramedullary canal.
